(19) United States
(12) Patent Application Publication
(10) Pub. No.: US 2020/0163655 A1
(43) Pub. Date: May 28, 2020

(54) Method for Collecting Ocular Secretions for Biomarker Diagnostics

(71) Applicant: Ascendant Diagnostics, LLC, Springdale, AR (US)

(72) Inventors: Lindsay Rutherford, Fayetteville, AR (US); Anna Daily, Fayetteville, AR (US); Omid Moghadam, Fayetteville, AR (US); Rutherford Micaheal, Fayetteville, AR (US)

(73) Assignee: Ascendant Diagnostics, LLC, Springdale, AR (US)

(21) Appl. No.: 16/686,346

(22) Filed: Nov. 18, 2019

Related U.S. Application Data
(60) Provisional application No. 62/771,208, filed on Nov. 26, 2018.

(51) Int. Cl. A61B 10/00; G01N 33/487; A61F 9/007; A61B 10/02

(52) U.S. Cl. CPC A61B 10/0045; G01N 33/487; A61B 2010/0067; A61B 10/02; A61F 9/00736

(57) ABSTRACT

The present disclosure is directed toward methods for obtaining and processing ocular fluid samples from a subject in preparation for further analysis for diagnostic purposes. Ocular fluid provides a superior sample medium for biomarker analysis as compared to other biological media such as blood, serum, saliva, urine or bodily secretions. Proper sample collection and processing is critical for further use as a diagnostic medium as studies have shown that biomarkers found in ocular fluid can degrade quickly if mishandled.

### BACKGROUND

The present application encompasses a methodology to collect, transport, store, and analyze ocular fluid samples enabling ocular fluid or tear based diagnostic assays for a wide range of medical conditions. The aforementioned assays utilize proteins/protein fragments, also known as biomarkers, in the ocular fluid sample to diagnose, monitor, and/or assist in the treatment of a wide range of medical conditions including, but not limited to cancer, mental illness, brain injury, multiple sclerosis, neurodegenerative disorders, and stroke.

Diagnostic tests are a key tool for the medical profession enabling diagnosis of a wide range of medical issues. These diagnostic tests have been critical in advancing medicine to the current state of the art. Traditionally, bodily fluids such as blood, saliva, semen, cerebrospinal fluid, serum, vaginal secretions, bile and urine have been used for diagnostic tests along with bodily tissue. (Doecke 2012, Madico 1998, Streckfus 2002) However, interferences inhibit or block identification and/or quantification of biomarkers in these sample mediums, especially blood. Diagnostics utilizing blood samples often also have concerns with hemolysis. (Chevillet 2014) Our work has shown that low molecular weight biomarkers are often obscured by larger proteins in blood samples as compared to an ocular fluid matrix. The present invention highlights a method to enable the use of ocular fluids, such as tears, to be used as a diagnostic medium for a wide range of medical conditions.

### SUMMARY

Methods of collecting and processing ocular fluid samples taken from a subject are provided herein. This methodology for the collection and processing of ocular fluid samples for use in medical diagnostic testing enables the use of biomarkers found in ocular fluid to be reliably used for the diagnosis of a variety of medical conditions including, but not limited to, cancer, brain trauma, mental illness, neurodegenerative disorders, and multiple sclerosis. Appropriate post collection treatment of ocular fluid samples is critical for their use as a diagnostic medium. The outlined methodology includes utilizing a sample collector to obtain an ocular fluid sample from a subject, prepare the sample for transport or storage, and further prepare the sample for diagnostic analysis. Kits for performing methods described herein along with medical devices capable of performing the diagnostic testing outlined are also provided herein.

### BRIEF DESCRIPTION OF THE DRAWINGS

So that those skilled in the art to which the present invention relates will readily understand how to make and use the present invention without undue experimentation, preferred embodiments thereof are described in detail below with reference to certain FIGURES in which:

FIG. 1: Flow diagram of sample collection methodology.

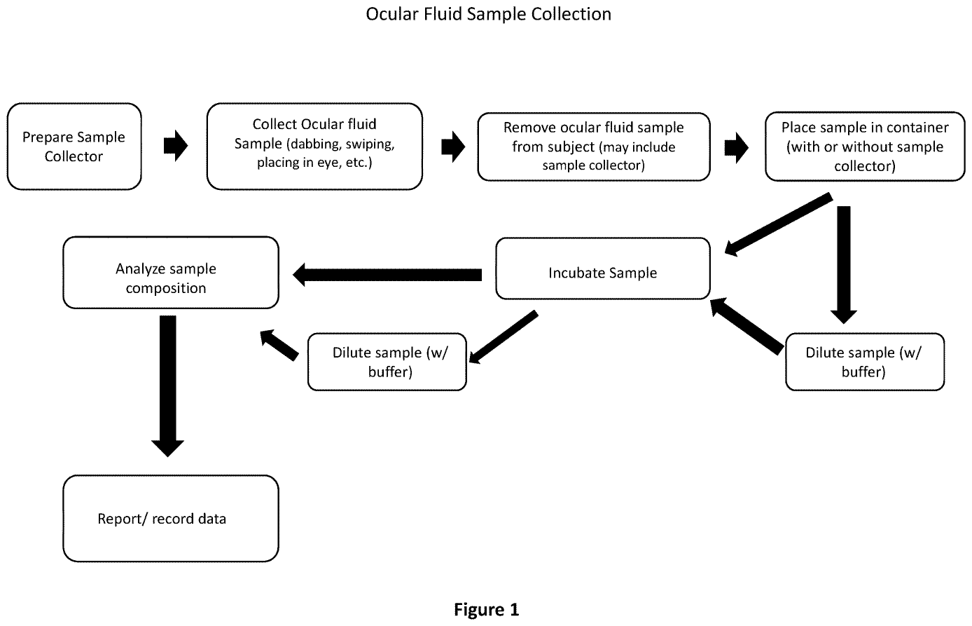

### DETAILED DESCRIPTION

Provided herein are a method and a collection kit required to utilize ocular fluid based biomarkers in diagnostic medicine. The methodology used to collect ocular fluid samples from a patient is a critical step allowing successful utilization of proteins and protein fragments, collectively termed biomarkers, as a diagnostic medium. Without proper sample collection and sample handling, the performance of ocular fluid based diagnostics is diminished significantly through degradation of target biomarkers after collection. Work has shown prolonged residency on the sample collector leads to diminished usability of several biomarkers found in ocular fluid. A representative sample of this phenomenon is shown in Table 1. The observed degradation occurs at conditions generally thought to be favorable to biomarker storage (i.e. low temperature in controlled environment), thus exposure to ambient conditions may shorten the post collection lifetime of the samples. The biomarker shown in table 1 is a representative biomarker highlighting the phenomenon observed across a wide range of biomarkers of interest in ocular fluid based diagnostics. This negative trend highlights the need for appropriate sample collection and post collection treatment in order to ensure that the biomarkers of interest in ocular fluid remain viable for quantification for a suitable period of time required for analysis. The suitable period of time required for analysis depends on a number of factors including geographic proximity to a laboratory suitable for analysis and conditions the sample may encounter in transit

TABLE 1

| | | | | | | Fold | Percent |
|---|---|---|---|---|---|---|---|
| | Initial | 7 days | 28 days | 84 days | 365 days | Change | Change |
| Subject 1 | 2.27E+05 | 1.49E+05 | | | | 0.66 | 34.1 |
| Subject 2 | 3.51E+05 | 4.87E+04 | | | | 0.14 | 86.1 |
| Subject 3 | 7.55E+04 | 1.89E+05 | | | | 2.50 | 150.2 |
| Subject 4 | 9.66E+05 | 4.05E+05 | | | | 0.42 | 58.1 |
| Subject 5 | 3.37E+05 | 1.93E+05 | | | | 0.57 | 42.6 |
| Subject 6 | 2.02E+03 | | 5.67E+03 | | | 2.80 | 179.9 |
| Subject 7 | 3.12E+05 | | 6.27E+04 | | | 0.20 | 79.9 |
| Subject 8 | 6.13E+05 | | 1.25E+05 | | | 0.20 | 79.6 |
| Subject 9 | 8.31E+05 | | 1.78E+05 | | | 0.21 | 78.6 |
| Subject 10 | 2.41E+05 | | 4.44E+04 | | | 0.18 | 81.6 |
| Subject 11 | 1.06E+05 | | | 4.11E+04 | | 0.39 | 61.2 |
| Subject 12 | 1.22E+05 | | | 6.78E+04 | | 0.56 | 44.4 |
| Subject 13 | 1.32E+05 | | | 2.65E+05 | | 2.00 | 100.4 |
| Subject 14 | 4.54E+05 | | | 2.42E+06 | | 5.34 | 433.5 |
| Subject 15 | 4.25E+04 | | | | 6.82E+03 | 0.16 | 84.0 |
| Subject 16 | 4.89E+05 | | | | 1.14E+05 | 0.23 | 76.6 |
| Subject 17 | 1.80E+05 | | | | 5.40E+04 | 0.30 | 70.0 |
| Subject 18 | 3.58E+05 | | | | 6.77E+04 | 0.19 | 81.1 |
| Subject 19 | 4.81E+05 | | | | 3.18E+04 | 0.07 | 93.4 |

to said laboratory. Biomarkers found in ocular fluid, if collected and stored properly, can be used to determine the disease state of a patient or other subject.

Ocular fluid, as referenced throughout this disclosure, is defined as any fluid obtained from any surface of the eye or ocular cavity containing analytes of interest, including protein, DNA, RNA, bacteria, viruses, biomarkers, etc.) which may have arisen within the ocular cavity or at another location within the body. Examples of ocular fluid comprise lacrimal secretions, vitreous humor, aqueous humor, meibum, and tears. The terms “ocular fluid”, “ocular wash”, “tear(s)” and “ocular secretion” are used interchangeably.

Subjects include humans, livestock, domesticated animals such as cats, dogs, cows, pigs, or other animals susceptible to cancer, injury, or disease; or is being tested for having cancer, injury, or disease. The terms subject and patient are used interchangeably herein. The subjects can be suspected of having a medical condition, being treated for a medical condition, or being monitored post treatment for a medical condition. The methods and kits described herein can be used to diagnose, monitor, and prevent the spread of disease and other medical conditions. “Medical condition” as used throughout is defined as any condition requiring diagnosis or treatment by a medical professional. Examples are, but not limited to: Cancers, AIDS, mental health disorders, brain trauma, neurological disease, communicable diseases, sexually transmitted diseases, autoimmune disease, liver disease, kidney disease, parasite transferred disease, and substance abuse among others. cancers include, but are not limited to: breast cancer, acoustic neuroma, acute lymphoblastic leukemia, acute myelogenous leukemia, adrenal tumors, AIDS-associated cancers, basal cell carcinoma, benign blood disorders, bladder cancer, bone cancer, brain tumors (metastatic and primary), breast cancer, cancer of unknown primary origin, cervical cancer, chronic lymphocytic leukemia, chronic myelogenous leukemia, colorectal cancer, esophageal cancer, gallbladder and bile duct cancers, gastrointestinal neuroendocrine tumors, GERD, Barrett's esophagus and achalasia, gestational trophoblastic disease, head and neck cancers, Kaposi sarcoma, kidney cancer, leukemias, liver cancer, liver metastases, low-grade glioma, lung cancer, lymphoma, male breast cancer, melanoma, Merkel cell carcinoma, mesothelioma, multiple myeloma, myelodysplastic syndrome, ovarian cancer, pancreatic cancer, pancreatic cysts, pituitary tumors, prostate cancer, pulmonary neuroendocrine tumors, rare blood disorder, skin cancer, soft tissue sarcoma, spine tumors, squamous cell carcinoma, stomach (gastric) cancer, testicular cancer (germ cell tumors), thymoma and other thymic tumors, tracheal diseases, uterine (endometrial) cancer, uterine sarcoma. Neurodegenerative disorders include but are not limited to: Alzheimer's disease, Amyotrophic lateral sclerosis (ALS), Dementia, Friedreich's ataxia, Huntington's disease, Lewy Body Disease, Motor neuron disease, Parkinson's disease, Parkinson's disease related disorders, Prion disease, Spinocerebellar ataxia (SCA), and Spinal muscular atrophy Mental health disorders include, but are not limited to: Amphetamine Dependence, Anorexia nervosa, Anxiety disorder, Asperger's Disorder, Attention-deficit hyperactivity disorder (ADHD), Autism spectrum disorder, Binge Eating Disorder, Bipolar Disorder, Borderline Personality disorder, Bulimia Nervosa, Cognitive disorders, Delirium, Dementia, Depression, Dissociative Amnesia, Dissociative Disorders Dissociative Identity Disorder, Dyspareunia, Dyssomnias, Erectile Disorder, Generalized Anxiety disorder, Impulse Control Disorder, Major Depressive Disorder (MDD), Obsessive-compulsive disorder (OCD), Panic Disorder, Parasomnias, Personality Disorders, Pervasive Developmental Disorder, Postpartum Depression, Posttraumatic Stress Disorder (PTSD), Psychosis, Schizophrenia, Seasonal Affective Disorder, Social Anxiety, Somatoform Disorders, substance abuse, and Tourette's disorder.

In one embodiment, the method of sample collection and preservation outlined are used to collect and prepare a subject's ocular fluid sample for analysis within a physician's office, subject's home or place of work, or another laboratory for further analysis. The sample is collected by placing a sample collection device in a single or both eyes of the subject for a pre-determined amount of time. After the time has elapsed the device is removed from the subject's eye and placed in a vial containing a liquid buffer solution. The sample is left, or incubated, in the buffer solution until further sample processing is warranted. Prior to running a diagnostic assay, the sample containing vial may be placed in a centrifuge to expedite removal of proteins from the sample collector. The sample can then be further diluted if need be and used for a diagnostic test.

In another embodiment, the sample collection device is an Ophthalmic Diagnostic Strip, such as a Schirmer Tear Flow Test Strip. The Schirmer strip is removed from the packaging and the end is folded over (forming on or about a 45° angle) prior to placement of the folded portion in between the lower eyelid (conjunctiva) and the surface of the eye (sclera) of the subject. The subject is directed to close the eye while the Schirmer strip is in place and to keep the eye closed for the duration of time the sample collector is in the eye. The ocular fluid collection device is left in place until either the ocular fluid reaches the 25 mm marking on the Schirmer strip (or other desired marking) or the desired collection time has elapsed. Generally the desired collection time is between 10 seconds and 10 minutes, preferably 5 minutes. After removal from the eye, the collection device is placed in a sample vial containing a volume of buffered solution. The buffered solution is preferably sterile Phosphate Buffered Saline (PBS) with a pH of 6.8. The collection device is incubated in the sample vial for a period of time ranging from 1 min to indefinite, but preferably 3 to 30 hours. The post collection incubation is performed at ambient temperature. The post collection incubation may also be performed at any desired temperature ranging from −90° C. to 99° C. After incubation the sample containing vial is placed in a centrifuge and spun between 5,000 to 10,000 rpm. The liquid is removed from the vial and used for further analysis or placed in storage. Further analysis is a diagnostic assay to quantify or detect disease, injury, or disorder specific biomarkers.

In yet another embodiment, the ocular fluid sample is collected using a device wherein the surface of the collection device is comprised of or covered with a fabric covering. The fabric covering can be comprised of natural fibers, such as cotton, or synthetic material, such as Rayon, Dacron, or Nylon. The selection of fabric covering is based on the physical interactions of the fabric and the biomarkers of interest. It is preferred that the fabric collect the biomarkers of interest from the subject but release them when desired for further analysis (i.e. release biomarkers of interest into the solution inside the sample vial during incubation). Examples of sample collecting devices include cotton swabs, culture swabs, and versions of such covered with a natural or synthetic fiber material commonly used in medical environments. To obtain the ocular fluid sample, the sample collector is contacted to the inner portion of the eye lid (conjunctiva) and/or the surface of the subject's eye (sclera) with the fabric coated surface. The sample collector's contact with the subject is either dabbed or swiped briefly to absorb or collect the ocular fluid. Preferably the sample collector is dabbed 3-5 times on the lower eye lid. After collecting the ocular fluid the sample collector is placed in a liquid filled vial in which the volume of liquid is sufficient to cover the fabric coated portion of the sample collector.

In an additional embodiment, the ocular fluid sample is collector by placing a piece of filter paper between the subject's eye and eye lid. In addition to filter paper, nitrocellulose paper or membrane can also be used. An example of filter paper suitable for collecting ocular fluid samples is Whatman No. 41. The paper based sample collector is removed from the eye after a period of time then placed in a fluid containing vial containing sufficient liquid to cover the entire piece of sample collecting paper. The paper may be pushed into the vial and compressed using an external force or tool if needed to ensure the entirety of the sample is covered by the liquid inside the vial. Preferably the liquid inside the vial is water, or buffer solution. The sample is then incubated for a period of time. Post incubation the sample is centrifuged and the sample containing supernatant is decanted. Further analysis or sample dilution can be carried out prior to use in a diagnostic assay.

In yet another embodiment the ocular fluid sample is placed into a sample vial containing a volume of liquid sufficient to contact or cover the sample collector. Specifically, if the volume of the vial is rated for 1000 ul, then the volume added to the tube is 225-250 ul. The fluid inside of the sample vial is a pH buffered phosphate saline solution, commonly termed PBS buffer. Other buffers suitable for ocular fluid biomarker preservation can be taken from the list comprising: Tris buffer, Hepes Buffer, Saline based buffers, saline sodium citrate buffers, MOPS buffer, or other suitable biological buffer system. Further the buffer may contain one or more of the following protease inhibitor cocktails: PMSF, Benzamidine, pepstatinA, Leupeptin, Aprotinin, EDTA, EGTA, AEBSF, E-64 cysteine protease inhibitor, Bestatin, PhosSTOP™, DMSO, Phosphoramidon disodium salt, Elastatinal, Nafamostat meylate, Okadaic acid, Sodium fluoride, Sodium orhtrovanadate, Bromotetramisdole oxalate, Beta lactose, DL leucine, trehalose, StabilZyme®, StabilCoat®, and/or StabilGuard®. A preservative, surfactant, or additive may or may not also be added to the liquid in the tube based on the compatibility of the biomarkers of interest with the sample collector, sample collector covering, and incubation conditions. Suitable preservatives, additives, and surfactants comprise: Polysorbate 20, Polysorbate 40, Polysorbate 80, Polysorbate 85, glycerol, purified bovine serum albumin (BSA), sodium azide, ethylene glycol, Phenylmethlsulfonyl fluoride and/or sodium chloride. Any additive or preservative agent is added in a weight percentage of 0.5% to 30% based on the characteristics of the biomarkers of interest and the physical properties of the sample collector material used, and the projected incubation conditions.

In another embodiment, the invention provides, inter alia, a method of collecting and preparing ocular fluid samples for diagnostic analysis wherein after the ocular fluid samples are collected using a sample collection device, the sample collection device containing the ocular fluid sample, is placed into a liquid medium inside a small container, such as a sample vial, and then incubated for a period of time prior to further handling or use. This incubation can be performed at ambient temperatures, however incubation at lower temperatures such as 0° C., 4° C., or −80° C. is suitable based on the biomarkers of interest. The duration of incubation can be between 0.5 hour and 2 months with a 6-36 hour incubation preferred. The incubation allows for the ocular fluid sample to elute into the liquid contained in the vial. After incubation, the ocular fluid samples are allowed to acclimate to ambient conditions prior to further processing in preparation for analysis.

In even another embodiment, the method outlined for the collection of ocular fluid samples and further transport or processing of said ocular fluid samples utilizes a kit containing the necessary supplies to collect said ocular fluid samples from a subject. The kit shall be enclosed in a suitable container as to contain all of the needed supplies for collection of ocular fluid samples from a subject. The container may be made of cardboard, paper, plastic or recycled versions of the previously outlined materials. Preferably the kit is housed in a cardboard box-like container. The kit is comprised of: protective gloves (such as latex or nitrile), devices for collecting ocular fluid from the subject's eye, sample vial, suitable buffer solution, device for manipulating sample collector once placed in sample vial, sample transport tracking information, sample identification labels, and disposal vessel. Such a kit is suitable for use in a doctor's office, medical clinic, subject's place of work or living, and temporary versions of each.

In the one embodiment, the sample collection kit is comprised of a cardboard, resealable plastic bag, or resealable container whose contents comprise: a single pair of nitrile gloves, two Ophthalmic Diagnostic Strips (also known as Schirmer Tear Flow Test Strips), a rod for manipulation of sample collection device in the sample vial, vial containing a suitable buffer solution, and patient identification labels. The manipulation rod is preferably composed of a non-porous material such as plastic, however a wooden rod is also suitable. The sample vial can be comprised of any fluid tight vessel with an internal volume sufficient to hold the sample collection device and appropriate liquid to maintain contact with the sample collection device. An example of a suitable collection vessel is a 1.5 ml conical micro centrifuge tube (such as Eppendorf 022363204), or a 0.7 ml external threaded pre-barcoded sample vial (such as FluidX 68-0703-11 or similar). The preferred sample vial should have a unique sample identification pre-printed, able to be applied, on the outer surfaces of the vial such as a barcode, 2D barcode, or other human or non-human readable identification system.

### Example 1

### Stability Evaluation of Biomarkers in Ocular Fluid Samples Vs Time

Two ocular fluid samples were collected from a each subject (one sample from each eye) using an Ophthalmic Diagnostic (Schirmer) Strip sample collector in a single sitting. Immediately following collection one of the samples, Sample A, was placed into a buffer solution and placed on a laboratory shaker (100 rpm) at 4° C. for three hours. After the incubation period had elapsed, the sample collection strip was removed. The remaining solution was analyzed for biomarkers of interest using ELISA. ELISA assays were conducted immediately with the remaining sample placed in an ultra-low freezer (−80 C) until further use.

The sample collector from the second eye of the subject, (Sample B), was placed in a 1.5 ml microcentrifuge tube without a buffer solution and immediately placed in the ultra low freezer (−80 C) until further use.

Sample B from each subject was removed from the ultra-low freezer allowed to acclimate to ambient temperature prior to further processing. A buffer solution was added to the microcentrifuge tube containing the sample collector and subsequently placed on an orbital shaker at 4° C. for a period of three hours at 100 rpm. The samples were then removed, aliquoted, and placed back into the ultra-low freezer until analysis. Sample B was removed from the freezer along with Sample A. Samples were thawed at room temperature and prepared for ELISA. Samples A and B were run concurrently using an ELISA assay with the results compared back to the original ELISA analysis (prior to freezing the sample) Further analysis using the B samples was conducted at various intervals, including 1 week, 4 week, 12 week, and I year, to monitor for biomarker degradation.

The preceding discussion and examples have been given for understanding and clarity purposes. No unnecessary limitations are to be understood from them. The disclosed invention is not to be limited to the exact details shown and described, for variations obvious to one skilled in the art will be included within the invention defined by the following claims.

All numbers expressing quantities of components, molecular weights, percentages, and so forth used in the specification and claims are to be understood in all instances by the term “about” unless otherwise stated. Numerical parameters set forth in the specification and claims are approximations that may vary depending upon the properties desired to be obtained by the present invention unless otherwise indicated.

The term “and/or” means one or all of the listed elements or a combination of any two or more of the listed elements. The term “Comprises” and variations of thereof do not have a limiting meaning where these terms appear in the claims or description unless otherwise specified. The terms “a”, “an”, “the”, and “at least one” are used interchangeably and reference more than one. All references or recitations of numerical ranges by endpoints include all numbers within that range (e.g. 1 to 4 includes 1, 1.1, 1.75, 2, 2.4, 3, 3.6, 4, etc).

Any and all headings are merely for the convenience of the reader and should not be used to limit the meaning of the test following the heading unless otherwise specified as such.

It will be appreciated by those persons skilled in the art that variations and/or alterations could be made to the invention without varying from the scope or spirt of the present inventions described broadly. The presented embodiments are to be considered in all aspects as illustrative and not restrictive. All references cited herein are incorporated by reference to the maximum extent allowable by law.

### REFERENCES

Chevillet, J., Lee, I., Briggs, H., He, Y., & Wang, K. (2014). Issues and prospects of microRNA-based biomarkers in blood and other body fluids. Molecules, 19(5), 6080-6105.
Doecke, J. D., Laws, S. M., Faux, N. G., Wilson, W., Burnham, S. C., Lam, C. P., & De Ruyck, K. (2012). Blood-based protein biomarkers for diagnosis of Alzheimer disease. Archives of neurology, 69(10), 1318-1325.
Madico, G., Quinn, T. C., Rompalo, A., McKee, K. T., & Gaydos, C. A. (1998). Diagnosis of Trichomonas vaginalis Infection by PCR Using Vaginal Swab Samples. Journal of clinical microbiology, 36(11), 3205-3210.
Streckfus, C. F., & Bigler, L. R. (2002). Saliva as a diagnostic fluid. Oral diseases, 8(2), 69-76.

What is claimed:

1. A method for collecting ocular fluid from a subject for use in diagnostic assays comprising the steps of:
a. Placing or touching a sample collection device in or to the surface of a subjects eye,
b. Removing said sample collection device containing ocular fluid and placing said sample collection device, and ocular fluid sample into a container,
c. incubating said container for a period of time,
d. centrifuging said sample container for a period of time,
e. removing the ocular fluid sample from container and using for diagnostic purposes.

2. The ocular fluid as in claim 1 is comprised of tears, lacrimal secretions, aqueous humor, vitreous humor, and meibum.

3. The method of claim 1 wherein the surface of the subject's eye is comprised of at least the Cornea, Sclera, and Conjunctiva.

4. The method of claim 1 wherein the sample collector is placed, rubbed, or touched to the subject's eye, both eyes, or multiple sample collectors are placed in a single or both eyes of the subject.

5. The method of claim 1 wherein the sample collector is an Ophthalmic diagnostic strip, piece of filter paper, cotton swab, culture swab, device covered with a natural or synthetic fiber comprised of rayon, nylon, dacron, Kevlar, or polyurethane

6. The method of claim 1 wherein the sample collector is touched to the surface of the subject's eye, conjunctiva, cornea, or sclera multiple times.

7. The method of claim 1 wherein the sample collector is swiped across the subject's conjunctiva, cornea, or sclera

8. The method of claim 1 wherein a portion or entirety of the sample collector is placed in between the subject's conjunctiva and sclera or cornea for a period of time.

9. The method of claim 8 where in the period of time is between 5 seconds and 10 minutes.

10. The method of claim 1 where in the entirety or the portion of the sample collector having come into contact with the eye is placed into said container.

11. The method of claim 1 wherein said container is liquid tight

12. The method of claim 1 wherein said container is labeled with sample identification information.

13. The method of claim 1 wherein the sample collection device is placed in said container and manipulated using a rod or conical shaped device.

14. The method of claim 1 wherein the sample is incubated in said vial for a period of 5 minutes to 5 days.

15. The method of claim 1 wherein the sample is incubated in the vial for a period of 12 to 36 hours.

16. The method of claim 1 wherein the sample is incubated in the vial at ambient condition

17. The method of claim 1 wherein the sample is incubated in the vial at a temperature ranging from −80° C. to 4° C.

18. The method of claim 1 wherein said ocular fluid sample in said container is incubated in said container at an elevated temperature ranging from 22° C. to 100° C.

19. The method of claim 1 wherein said sample is centrifuged in said container for a period of time ranging from 10 seconds to 10 minutes.

20. The method of claim 1 wherein said diagnostic purpose is to diagnose, treat, monitor the treatment of, or monitor reoccurrence of a medical condition.

21. The medical condition of claim 20 is chosen from a list comprising: Cancer, AIDS, mental health disorders, brain trauma, neurological disease, communicable diseases, sexually transmitted diseases, autoimmune disease, liver disease, kidney disease, parasite transferred disease, neurodegenerative disease, and substance abuse

22. The method of claim 1 wherein said container contains a liquid solution.

23. The method of claim 1 wherein a liquid is added to said container after the sample collector is placed into said container.

24. The liquid solution of claim 22 wherein said liquid is water, buffer solution, or aqueous solution comprised of at least one protease inhibitor, preservative agent, additive, or surfactant.

25. The buffer solution of claim 24 where in the buffer is chosen from the list comprising Tris buffer, HEPES Buffer, Saline based buffers, saline sodium citrate buffers, MOPS buffer

26. The protease inhibitor of claim 24 wherein said agent is at least PMSF, Benzamidine, pepstatinA, Leupeptin, Aprotinin, EDTA, EGTA, AEBSF, E-64 cysteine protease inhibitor, Bestatin, PhosSTOP™, DMSO, Phosphoramidon disodium salt, Elastatinal, Nafamostat Meylate, Okadaic acid, Sodium fluoride, Sodium orhtrovanadate, Bromotetramisdole oxalate, Beta lactose, DL leucine, trehalose, StabilZyme®, StabilCoat®, or StabilGuard®

27. The preservative, additive or surfactant of claim 24 wherein said agent is at least Polysorbate 20, Polysorbate 40, Polysorbate 80, Polysorbate 85, glycerol, purified bovine serum albumin (BSA), sodium azide, ethylene glycol, Phenylmethlsulfonyl fluoride or sodium chloride

28. The method of claim 1 wherein the diagnostic purposes is a diagnostic test.

29. The diagnostic test of claim 28 where in the test is an immunological assay, lateral flow device, and/or laboratory developed test

30. The method of claim 1 wherein the subject is human

31. The method of claim 1 wherein the subject is an animal.

32. A kit to be used for the collection of ocular fluid samples comprised of a ocular fluid sample collector, aqueous solution, sample container, sample identification markings, and patient identifying information.

33. The kit of claim 32 where the contents of the kit are contained in a single vessel

34. The kit of claim 32 where the sample collector and container are a single unit.

35. The kit of calm 32 where the aqueous solution is a buffer selected from the list comprising: Tris buffer, Hepes Buffer, Saline based buffers, saline sodium citrate buffers or, MOPS buffer

36. The kit of claim 32 where the aqueous solution contains a preservative, surfactant or additive taken from the list comprising: Polysorbate 20, Polysorbate 40, Polysorbate 80, Polysorbate 85, glycerol, purified bovine serum albumin (BSA), sodium azide, ethylene glycol, Phenylmethlsulfonyl fluoride and/or sodium chloride.

37. The kit of claim 32 wherein the aqueous solution is sterile.

38. The kit of claim 32 where in the aqueous solution is comprised of a buffer, surfactant, preservative, and de-ionized water.
